(19) United States
(12) Patent Application Publication
(10) Pub. No.: US 2025/0049453 A1
(43) Pub. Date: Feb. 13, 2025

(54) ENDOSCOPIC TUBE

(71) Applicant: HUKUI BIOTECHNOLOGY CO., LTD., Taipei City (TW)

(72) Inventors: SHIH-CHE LO, Taipei City (TW); YI-MING CHU, Taipei City (TW); MING-KUN CHAN, Taipei City (TW)

(73) Assignee: HUKUI BIOTECHNOLOGY CO., LTD., Taipei City (TW)

(21) Appl. No.: 18/795,212

(22) Filed: Aug. 6, 2024

Related U.S. Application Data
(60) Provisional application No. 63/517,934, filed on Aug. 7, 2023.
(60) Provisional application No. 63/589,998, filed on Oct. 13, 2023.

(30) Foreign Application Priority Data
Jun. 6, 2024 (TW) 113121050

(51) Int. Cl. A61B 17/22; A61B 1/00; A61M 25/10

(52) U.S. Cl. CPC A61B 17/22; A61B 1/00082; A61M 2025/1068

(57) ABSTRACT

An endoscopic tube includes: a body; a catheter connected to the body, having therein one single working channel, having a proximal opening proximal to the body, and having a distal opening distal to the body; and an inflation device enclosing the catheter, having an expansion free end proximal to the distal opening of the catheter and made of an elastic material, having one end proximal to the distal opening of the catheter and being closed, and having one end proximal to proximal opening of the catheter and being in communication with a gas admitting component having a gas admitting opening for admitting an external fluid, and thus the expansion free end expands when a gas is introduced into a space between the inflation device and the catheter through the gas admitting opening. The endoscopic tube is suitable for observing, sampling and/or treating pulmonary lesions.

### CROSS-REFERENCE TO RELATED APPLICATION

This non-provisional application claims priority under 35 U.S.C. § 119(a) on Patent Application No. 113121050 filed in Taiwan, R.O.C. on Jun. 6, 2024, the entire contents of which are hereby incorporated by reference.

This non-provisional application claims priority under 35 U.S.C. § 119(e) on US provisional Patent Application No. 63/517,934 filed on Aug. 7, 2023 and US provisional Patent Application No. 63/589,998 filed on Oct. 13, 2023, the entire contents of which are hereby incorporated by reference.

### BACKGROUND OF THE INVENTION

### 1. Field of the Invention

The present disclosure relates to endoscopic tubes, and in particular to an endoscopic tube comprising a catheter having one single working channel.

### 2. Description of the Related Art

Endoscopic tubes are for use in observing, sampling and/or treating pulmonary lesions. Conventionally, a catheter of an endoscopic tube must have at least two channels in order to observe a lesion while sampling and/or treating a lesion. An image capturing device is disposed in one of the channels, and a working element (for example, a sampling device or rinsing device) is disposed in the other channel.

Designed to have at least two channels, the catheter of a conventional

endoscopic tube has a dimension that typically comes with a lower limit of 3 mm, preventing the catheter from reaching deep pulmonary lesions.

### BRIEF SUMMARY OF THE INVENTION

Conventional endoscopic tubes still have room for improvements.

Therefore, it is an objective of the disclosure to provide an endoscopic tube with a catheter being of a small diameter in order to reach deep pulmonary lesions.

To achieve the above and other objectives, the disclosure provides an endoscopic tube, comprising: a body; a catheter connected to the body, having therein one single working channel, having a proximal opening proximal to the body, and having a distal opening distal to the body; and an inflation device enclosing the catheter, having an expansion free end proximal to the distal opening of the catheter and made of an elastic material, having an end proximal to the distal opening of the catheter and being closed, and having an end proximal to proximal opening of the catheter and being in communication with a gas admitting component having a gas admitting opening for admitting an external fluid, wherein the expansion free end expands when a gas is introduced into a space between the inflation device and the catheter through the gas admitting opening.

In an embodiment of the disclosure, at least a portion of the catheter is near the distal opening and is made of a material not penetrable by X-ray.

In an embodiment of the disclosure, the endoscopic tube further comprises: a distal bending mechanism connected to the body and the catheter and comprising: an operating handle disposed outside the body and rotatably connected to the body;

a spool disposed inside the body and connected to the operating handle; and a cable partially wound around the spool and having two ends connected to two sides proximal to the distal opening of the catheter respectively, wherein the operating handle can be rotated to drive the cable moving, causing a portion of the catheter to bend, with the bent portion being adjacent to the distal opening of the catheter.

In an embodiment of the disclosure, the endoscopic tube further comprises: a catheter rotating mechanism connected to the body and the catheter and comprising: a rotatable element connected to the catheter and rotatably connected to the body, wherein the rotatable element can be rotated to move the catheter, allowing the catheter to rotate.

In an embodiment of the disclosure, the endoscopic tube further comprises a sensing mechanism linked to the distal bending mechanism and the catheter rotating mechanism to sense a bending state of the catheter near the distal opening and a rotating state of the catheter.

In an embodiment of the disclosure, the catheter in the endoscopic tube further has a rinsing port proximal to the proximal opening.

In an embodiment of the disclosure, the catheter in the endoscopic tube has a bellow cross parallel structure proximal to the distal opening.

The endoscopic tube of the disclosure comprises a catheter having one single working channel and thereby effectively reduces the dimensions of the catheter, allowing the catheter to reach any lesion deep in the lung.

### BRIEF DESCRIPTION OF THE DRAWINGS

For the sake of clarity, the accompanying drawings are presented to show parts and components enlarged, omitted or schematically drawn. Furthermore, the dimensions of the parts and components shown in the accompanying drawings do not fully reflect their actual dimensions.

FIG. 1 is a schematic view of an endoscopic tube according to an embodiment of the disclosure.

FIG. 2 is a schematic view of the endoscopic tube viewed from another angle according to an embodiment of the disclosure.

FIG. 3 is a cross-sectional view of an expansion free end of an inflation device of the endoscopic tube according to an embodiment of the disclosure.

FIG. 4 is an exploded view of the endoscopic tube according to an embodiment of the disclosure.

FIG. 5 is a schematic view of how to operate the endoscopic tube according to an embodiment of the disclosure.

### DETAILED DESCRIPTION OF THE INVENTION

To facilitate understanding of the object, characteristics and effects of this present disclosure, embodiments together with the attached drawings for the detailed description of the present disclosure are provided.

Embodiment of the disclosure:

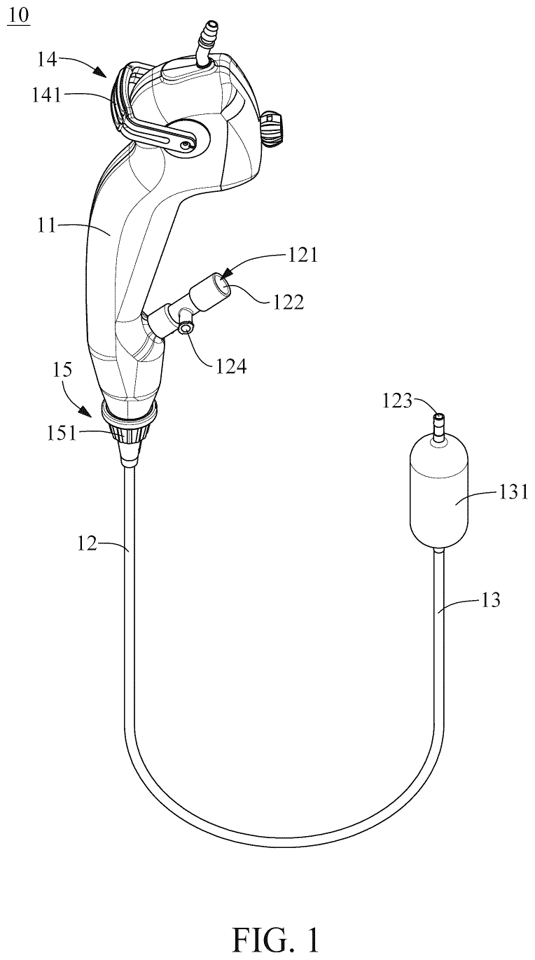

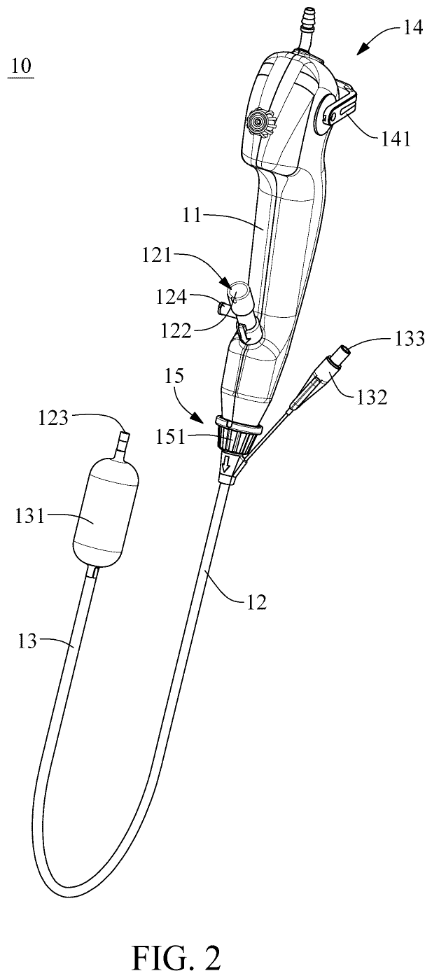

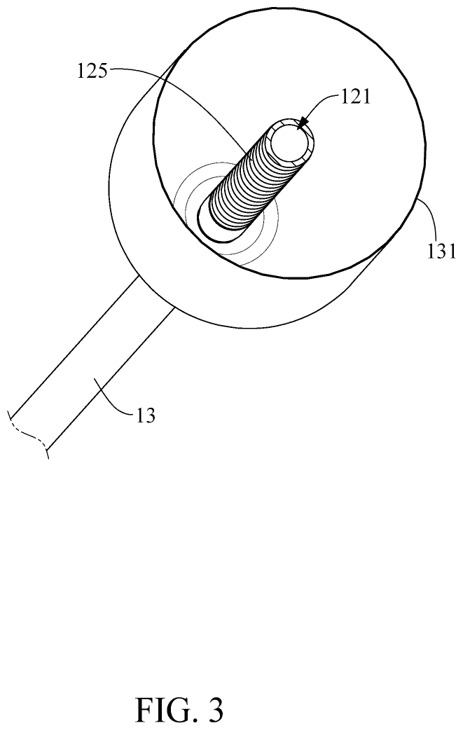

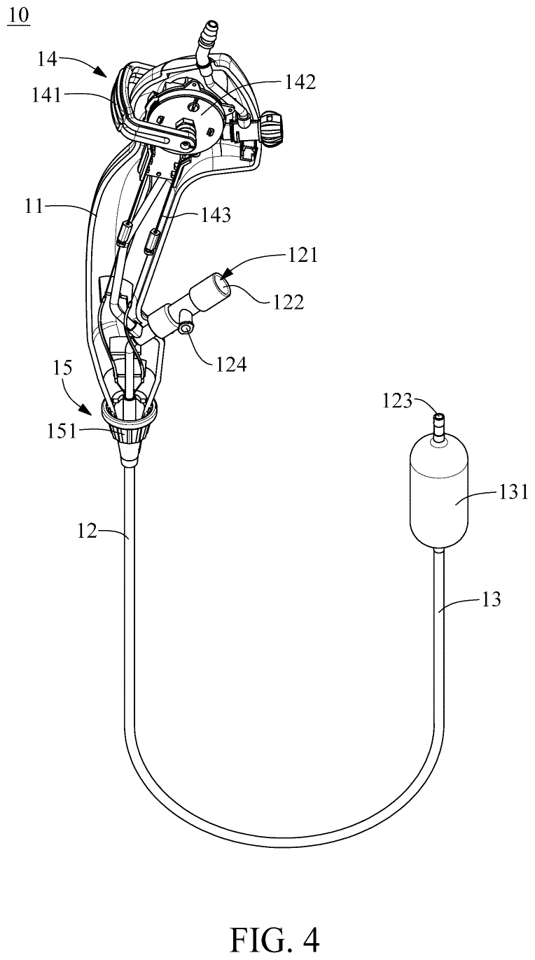

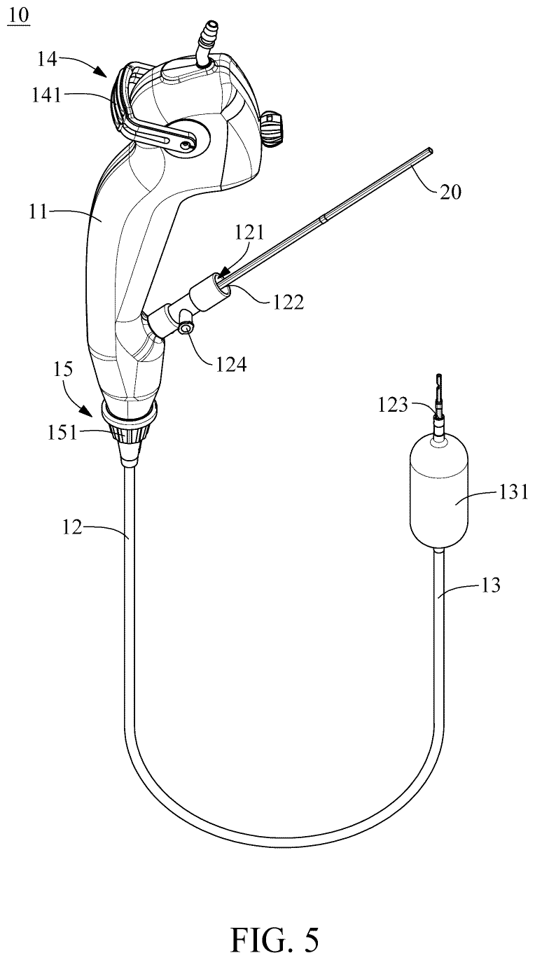

This embodiment provides an endoscopic tube shown in FIG. 1, FIG. 2, FIG. 3, FIG. 4 and FIG. 5.

In this embodiment, an endoscopic tube 10 comprises a body 11. For example, the body 11 is made of plastic or metal, but the disclosure is not limited thereto.

In this embodiment, the endoscopic tube 10 further comprises: a catheter 12 connected to the body 11, having a proximal opening 122 proximal to the body 11, and having a distal opening 123 distal to the body 11.

In this embodiment, the endoscopic tube 10 further comprises: an inflation device 13 enclosing the catheter 12, having an expansion free end 131 made of an elastic material and proximal to the distal opening 123 of the catheter 12, having one end proximal to the distal opening 123 of the catheter 12 and closed, and having one end proximal to the proximal opening 122 of the catheter 12 and being in communication with a gas admitting component 132 having a gas admitting opening 133 for admitting an external fluid.

The inflation device 13 has an expansion free end 131 is made of an elastic material and proximal to the distal opening 123 of the catheter 12. The expansion free end 131 expands (as shown in FIG. 1) when a gas is introduced into a space between the inflation device 13 and the catheter 12 through the gas admitting opening 133. When the gas is not introduced into a space between the inflation device 13 and the catheter 12, the expansion free end 131 is attached to the catheter 12 in order to penetrate the lung deep.

In this embodiment, the endoscopic tube 10 comprises the catheter 12 having one single working channel 121 and thereby effectively reduces the dimensions of the catheter 12 to less than 3 mm, allowing the catheter 12 to reach any lesion deep in the lung.

In this embodiment, the endoscopic tube 10 comprises the inflation device 13 such that, as soon as the distal opening 123 of the catheter 12 reaches a position near the lesion, the expansion free end 131 expands to fix the distal opening 123 in place or expands to dilate the bronchi, serving therapeutic purposes.

Referring to FIG. 5, in this embodiment, the endoscopic tube 10 comprises the catheter 12 having one single working channel 121 for containing a working element 20, and the working element 20 is an image capturing device, sampling device or rinsing device, but the disclosure is not limited thereto.

For example, in this embodiment, the image capturing device functioning as the working element 20 is placed in the working channel 121 and adapted to confirm that the distal opening 123 of the catheter 12 has already reached a position near the lesion, and then the expansion free end 131 expands to fix the distal opening 123 in place. At this point in time, since the positions of the distal opening 123 and the lesion relative to each other have already been fixed because of the expansion of the expansion free end 131, the user no longer needs to observe the lesion with the image capturing device; thus, the image capturing device in the working channel 121 can be withdrawn therefrom and replaced by the sampling device or rinsing device for sampling and/or treating the lesion.

In this embodiment, in the endoscopic tube 10, the expansion free end 141 expands to fix the position of the distal opening 123 such that the positions of the distal opening 123 and the lesion relative to each other are fixed to prevent the distal opening 123 from undergoing displacement relative to the lesion while the working element 20 is being changed.

In this embodiment, the endoscopic tube 10 further comprises a distal bending mechanism 14 connected to the body 11 and the catheter 12, but the disclosure is not limited thereto.

The distal bending mechanism 14 comprises: an operating handle 141 disposed outside the body 11 and rotatably connected to the body 11; a spool 142 disposed inside the body 11 and connected to the operating handle 141; and a cable 143 partially wound around the spool 142 and having two ends connected to two sides proximal to the distal opening 123 of the catheter 12 respectively. Therefore, the operating handle 141 can be rotated to drive the cable 143 moving, causing a portion of the catheter 12 to bend, with the bent portion being adjacent to the distal opening 123 of the catheter 12.

In this embodiment, the endoscopic tube 10 of an endoscopic tube set 1 further comprises a catheter rotating mechanism 15 connected to the body 11 and the catheter 12, but the disclosure is not limited thereto.

The catheter rotating mechanism 15 comprises a rotatable element 151 connected to the catheter 12 and rotatably connected to the body 11, and thus the rotatable element 151 can be rotated to move the catheter 12, allowing the catheter 12 to rotate.

The distal bending mechanism 14 and/or the catheter rotating mechanism 15 enables users to precisely control the orientation of the distal opening 123 of the catheter 12, allowing the distal opening 123 of the catheter 12 to reach any lesion deep in the lung.

In this embodiment, the endoscopic tube 10 further comprises a sensing mechanism (not shown) linked to the distal bending mechanism 15 and the catheter rotating mechanism 16 to sense the bending state of the catheter 12 near the distal opening 123 and the rotating state of the catheter 12, but the disclosure is not limited thereto.

The sensing mechanism enables users to monitor the bending state of the catheter 12 near the distal opening 123 and the rotating state of the catheter 12 in real time, allowing the distal opening 123 of the catheter 12 to reach any lesion deep in the lung.

In this embodiment, in the endoscopic tube 10, the catheter 12 further has a rinsing port 124 proximal to the proximal opening 122, but the disclosure is not limited thereto. Owing to the rinsing port 124, the endoscopic tube 10 rinses a lesion while the working element 20 is staying in the working channel 121, further enhancing the flexibility of the application of the endoscopic tube 10.

In this embodiment, in the endoscopic tube 10, the catheter 12 has a bellow cross parallel structure 125 proximal to the distal opening 123, but the disclosure is not limited thereto. Owing to the bellow cross parallel structure 125, a portion of the catheter 12 is near the distal opening 123 and has sufficient rigidity and bending capability, allowing the distal opening 123 of the catheter 12 to reach any lesion deep in the lung.

The invention is disclosed above by preferred embodiments. However, persons skilled in the art should understand that the embodiments are illustrative of the invention only, but shall not be interpreted as restrictive of the scope of the invention. Please note that all variations and replacements equivalent to the embodiments shall be deemed falling within the scope of the invention. Therefore, the legal protection for the invention shall be defined by the appended claims.

While the present disclosure has been described by means of specific embodiments, numerous modifications and variations could be made thereto by those skilled in the art without departing from the scope and spirit of the present disclosure set forth in the claims.

What is claimed is:

1. An endoscopic tube, comprising:
a body;
a catheter connected to the body, having therein one single working channel, having a proximal opening proximal to the body, and having a distal opening distal to the body; and
an inflation device enclosing the catheter, having an expansion free end proximal to the distal opening of the catheter and made of an elastic material, having an end proximal to the distal opening of the catheter and being closed, and having an end proximal to proximal opening of the catheter and being in communication with a gas admitting component having a gas admitting opening for admitting an external fluid,
wherein the expansion free end expands when a gas is introduced into a space between the inflation device and the catheter through the gas admitting opening.

2. The endoscopic tube of claim 1, wherein at least a portion of the catheter is near the distal opening and is made of a material not penetrable by X-ray.

3. The endoscopic tube of claim 1, further comprising:
a distal bending mechanism connected to the body and the catheter and comprising:
an operating handle disposed outside the body and rotatably connected to the body;
a spool disposed inside the body and connected to the operating handle; and
a cable partially wound around the spool and having two ends connected to two sides proximal to the distal opening of the catheter respectively,
wherein the operating handle can be rotated to drive the cable moving, causing a portion of the catheter to bend, with the bent portion being adjacent to the distal opening of the catheter.

4. The endoscopic tube of claim 3, further comprising:
a catheter rotating mechanism connected to the body and the catheter and comprising:
a rotatable element connected to the catheter and rotatably connected to the body,
wherein the rotatable element can be rotated to move the catheter, allowing the catheter to rotate.

5. The endoscopic tube of claim 4, further comprising a sensing mechanism linked to the distal bending mechanism and the catheter rotating mechanism to sense a bending state of the catheter near the distal opening and a rotating state of the catheter.

6. The endoscopic tube of claim 1, wherein the catheter further has a rinsing port proximal to the proximal opening.

7. The endoscopic tube of claim 1, wherein the catheter has a bellow cross parallel structure proximal to the distal opening.
